(19) United States
(12) Patent Application Publication
(10) Pub. No.: US 2023/0386456 A1
(43) Pub. Date: Nov. 30, 2023

(54) METHOD FOR OBTAINING DE-IDENTIFIED DATA REPRESENTATIONS OF SPEECH FOR SPEECH ANALYSIS

(71) Applicant: Novoic Ltd.

(72) Inventors: Jack Weston, Greater London (GB); Emil Fristed, London (GB)

(21) Appl. No.: 18/366,184

(22) Filed: Aug. 7, 2023

Related U.S. Application Data
(63) Continuation of application No. PCT/EP22/51452, filed on Jan. 24, 2022.

(30) Foreign Application Priority Data
Feb. 5, 2021 (EP) 21155636.0

(51) Int. Cl. G10L 15/18; G10L 15/06; G10L 15/05; G10L 19/032; G10L 15/16; G10L 25/66

(52) U.S. Cl. CPC G10L 15/1807; G10L 15/063; G10L 15/05; G10L 19/032; G10L 15/16; G10L 25/66

(57) ABSTRACT

The invention relates to a computer-implemented method of obtaining de-identified representations of audio speech data for use in a speech analysis task, the method comprising: pre-processing the audio speech data to remove timbral information; encoding sections of the pre-processed audio speech data into audio representations by inputting sections of the pre-processed audio data into a prosody encoder, the prosody encoder comprising a machine learning model trained using self-supervised learning to map sections of the pre-processed audio data to corresponding audio representations. The combination of removing timbral information during pre-processing and encoding segments of pre-processed audio data using an encoder trained using self-supervised learning results in the provision of strong prosodic representations which are substantially de-identified from the speaker.

### CROSS-REFERENCE TO RELATED APPLICATION(S)

This application is a bypass continuation of International Application No. PCT/EP2022/051452, filed on Jan. 24, 2022, which in turn claims priority to European Application No. 21155636.0, filed on Feb. 5, 2021. Each of these applications is incorporated herein by reference in its entirety for all purposes.

### TECHNICAL FIELD

The present invention relates to a computer-implemented method and system for obtaining data representations encoding speech for use in speech analysis tasks, in particular for monitoring or diagnosis of a health condition. More particularly, the invention relates to a machine learning method for encoding prosodic, i.e. non-linguistic, information within speech into data representations, usable for speech analysis tasks, which are de-identified from the speaker.

### BACKGROUND

There has been significant recent progress in developing machine learning systems for both spoken language analysis and understanding, and spoken language production. Applications in this field include automatic speech recognition, text-to-speech conversion, automated spoken language understanding tasks such as detecting sentiment, emotions and sarcasm and, particularly importantly, speech analysis for detecting and monitoring health conditions.

Information in speech is not only encoded in the words, sentences, and meaning it carries; there's rich information in the audio signal, other than the semantic information, that it is necessary to identify for many of the speech analysis tasks identified above. This acoustic, non-linguistic information can be used to infer complimentary information within speech: meanings of words can be changed, emotions conveyed, and the speaker recognised.

Taking the example of diarisation within the broad category of automated speech recognition, the task of separating different speakers could use semantic information within the speech to identify phrases commonly used to end a turn in conversation or when a question is posed. However the task is performed much more accurately where a system can understand non-linguistic content within the audio, for example to identify intonations and paralinguistic signalling indicating that a speaker has finished their turn.

Similarly, in the growing application of automated speech analysis within healthcare, there is rich information in the acoustic, non-linguistic, component of speech which is usable for the diagnosis and monitoring of a wide range of health conditions. Speech production is regulated by the interaction of a number of different physical, physiological, and psychological systems in the human body. At the higher levels it requires use of a number of different areas of the brain including those for memory to recall thoughts and concepts, the brain areas for sentence construction and word-recall in order to form the concepts into sentences and the brain areas that form phonetic representations to position and control of the vocal cord and other articulators of the articulatory system to control these organs to produce the required sounds for syllables and phonemes. Speech production is also dependent on these parts of the body themselves, including a healthy and correctly functioning vocal cord for correct positioning of the articulators and vocal folds, correct functioning of the articulatory system including timing and coordination of the articulators and vocal folds, a healthy and correctly functioning respiratory system for producing the airflow that's is converted into speech—and neural signalling that controls these system, for example for muscle activation.

There are a large range of diseases that impinge upon the correct functioning of these physiological systems resulting in changes to both choice of language but also non-linguistic components, for example the hesitations, pitch, tempo and rhythm. For example cognitive disorders such as Alzheimer's affect the brain and therefore impact on speech through both the higher-level speech systems such as memory but also the lower-level physiology in terms of the brain's ability to control the vocal cord and articulatory system.

Accordingly there is an ongoing need to extract representations of speech which encode non-linguistic information and can be used in speech analysis tasks.

This type of non-language acoustic information is prosody. Prosody is often defined substractively, for example as “the variation in speech signals that remains after accounting for variation due to phonetics, speaker identity, and channel effects (i.e. the recording environment)”. It can also be defined as the combination of timbre of speech (the spectral information which characterises a particular voice), the rhythm and tempo. Tempo relates to the speed and duration of voiced segments, while rhythm relates to the stress and intonation

There are a number of drawbacks in existing approaches to processing speech data to extract prosodic representations for use in machine learning based speech analysis tasks.

One significant issue is the difficulty in extracting prosodic representations which retain expressivity and encode all of the important non-linguistic information necessary for downstream speech analysis tasks, while being sufficiently de-identified from the speaker to protect user privacy and meet GDPR/HIPAA requirements. Much of the non-linguistic components of speech overlap with signals in the speech which are characteristic of the speaker. Human speech comprises a fundamental frequency F0 and spectral patterns at higher frequencies, such as the formant frequencies—the resonant frequency components in the vocal tract—which are characteristic of the speaker. A difficulty is that existing methods of encoding prosodic representations generally encode this characteristic information which can be used to identify the speaker. There is accordingly a need for a new approach which encodes prosody, without this speaker characteristic information.

A further issue is that prior art methods generally use a subtractive approach to encoding prosody. In particular, the methods often rely on conditioning an autoencoder model by requiring it to reconstruct input audio data through a data bottleneck, where the lexical information is provided to the model, encouraging it to learn representations which encode solely the non-lexical information. Such methods generally require significant pre-processing of the data, including the provision of the linguistic information, which is not always available. Furthermore, and more fundamentally, this approach to encoding prosody is not aligned with a human's natural interpretation of speech (where there is no parallel access to the lexical information when hearing speech) so may result in an incomplete encoding of the full prosodic information interpreted by the human brain as an unintended consequence of defining prosody in this way. There is accordingly a need for a new way of encoding prosody which has the possibility of capturing a greater proportion of the rich prosodic information present in human speech, in particular to promote the encoding of naturalistic prosody.

Generally prior art methods use heavily processed input speech data such as spectrograms or audio features which again, may result in a loss of information present in the raw audio. Attempts to process the raw audio directly are very computationally intensive and so there is a need for new methods which are able to utilise a greater proportion of the relevant information in the input audio signal to form prosodic representations but within current data processing limitations.

Accordingly there exists a need for a method of extracting prosodic representations of speech data for use in speech processing tasks which makes progress in overcoming the problems of the prior art. In particular there is a need for a method for extracting significantly de-identified representations, which encode as much of the prosodic information as possible in a computationally efficient manner.

### SUMMARY OF THE INVENTION

In one aspect of the invention there is provided a computer-implemented method of obtaining de-identified representations of audio speech data for use in a speech analysis task, the method comprising: pre-processing the audio speech data to remove timbral information; encoding sections of the pre-processed audio speech data into audio representations by inputting sections of the pre-processed audio data into a prosody encoder, the prosody encoder comprising a machine learning model trained using self-supervised learning to map sections of the pre-processed audio data to corresponding audio representations.

The combination of removing timbral information during pre-processing and encoding segments of pre-processed audio data using an encoder trained using self-supervised learning results in the provision of strong prosodic representations which are substantially de-identified from the speaker. In particular, a significant component of the speaker identifying information is removed during pre-processing so that this cannot be encoded in the representations. The model makes progress over prior art techniques which use subtractive models, requiring the model to be fed the linguistic content of the audio, and instead uses a new way of encoding prosody which has the possibility of capturing a greater proportion of the rich prosodic information present in human speech. The method therefore provides representations which are well suited to use in speech analysis tasks including automatic speech recognition, text-to-speech conversion, automated spoken language understanding tasks such as detecting sentiment, emotions and sarcasm and, particularly importantly, speech analysis for detecting and monitoring health conditions.

Preferably the method comprises encoding sections of the pre-processed audio speech data into quantised audio representations. Preferably the method comprises encoding sections of the pre-processed audio speech data into quantised audio representations, wherein the prosody encoder comprises a machine learning model trained to map sections of the pre-processed audio data to corresponding quantised audio representations. Forcing the model to encode the processed audio data into a fixed number of quantised states means the model must be parsimonious with what it chooses to represent and therefore is encouraged to encode solely the prosodic information, which can be used to make a prediction during training, and not the speaker identifiable information which is not predictive.

Quantised representations comprise discrete data representations with a fixed total number. In particular, rather than allowing a continuous feature space, the representations are restricted to a limited number of representations. A fundamental innovation described herein is the recognition that prosody forms a language with discrete prosodic “words” or units of language, which can be represented as quantised prosody representations (referred to here as quantised audio representations). This realisation allows the use of language models, originally developed for use with quantised linguistic representations, to learn quantised prosodic representations. This significantly departs from prior art techniques that use “subtractive” methods to lean prosody, by first trying to define and subtract the non-prosodic content (e.g. the linguistic content). The present method allows the model to learn what prosody is without having defining the information that should be learnt and encoded within the prosody representations.

Preferably pre-processing the audio data comprises applying a signal processing technique to remove speaker characteristic, preferably timbral, information, where the signal processing technique preferably comprises downsampling. In this way, the pre-processed audio data comprises a processed raw audio signal, preferably a downsampled raw audio signal. Sections of the processed raw audio are input into the machine learning model. This does not require firstly extracting features from this input as in prior art techniques but instead the machine learning model is provided with the processed raw audio signal directly. In some examples, pre-processing the audio data may also comprise training a machine learning model to remove speaker characteristic, preferably timbral, information from the audio data. For example, it may comprise inputting the audio data into an autoencoder conditioned with one or more components of the speech, for example the linguistic content or one or more components of prosody.

Timbral information comprises voice characteristics of the speaker (speaker-identifiable characteristics), in particular spectral characteristics of speech comprising the formant frequencies, which are the resonant frequency components in the vocal tract.

Preferably the audio speech data comprises a raw audio signal. In this way, the network is given full flexibility to learn what it wants as the full information present in the raw audio speech is provided to the model and no biases are introduced. Although this is preferred, in some examples the input audio speech data may comprise a spectrogram.

Preferably pre-processing the audio speech data comprises: downsampling the audio speech data at a rate of less than 1000 Hz, preferably between 400 Hz and 600 Hz, most preferably around 500 Hz. This ensures that the network is only learning about prosody not phonetics or other speaker identifiable characteristics which occur at higher frequency ranges. A further advantages is that this makes it possible to use longer sections of audio data as input and allow for word-length sections of audio which provide the relevant timescale range for extracting prosody most effectively.

Preferably the method comprises splitting the audio speech data into audio words, the audio words comprising variable length sections of the audio speech data, each containing one spoken word of the audio speech data; wherein the model is trained to map input audio words to corresponding quantised representations encoding prosodic information of the audio word. This provides stronger prosody representations as semantically meaningful prosody states are naturally discretized on a per-word basis. In this way the prosody encoder creates one independent representation per word.

Preferably the audio words comprise a period of silence preceding or following the spoken word. The phrase “a period of silence” is intended to refer to a period of the audio data which does not contain speech. Prosodic information is also present in non-spoken audio sections, for example in pauses, hesitations which influence rhythm. Including pre-ceding non-speech audio before a word allows the model to encode information relating to speech rate baseline and temporal variations, including absolute/relative speech rate.

Preferably the audio words comprise a period of silence preceding the spoken word wherein the period is up to 2 seconds in length. The non-spoken audio in the time preceding the word is more important than following it and may be more directly linked to the cognitive processes of the speaker and therefore is more preferable to encode in the representations.

Preferably the variable length audio words each have a length between 0.2 and 3 seconds, preferably 0.5 to 2 seconds. This provides sufficient time to encompass a word and any neighbouring period of silence.

Preferably the method comprises normalising the baseline pitch of audio speech data to a predetermined frequency. This may be provided within pre-processing or during the encoding stage, as part of the encoder model. In particular, the input data may be pitch shifted such that the median pitch of voiced segments is the same across speakers. This reduces the amount of baseline pitch information represented within the representations, making them less identifiable. Furthermore baseline pitch is not part of prosody, so it allows stronger representations to be formed which only encode variations form the normalised baseline frequency.

Pre-processing the input audio speech data preferably comprises removing sections of audio speech data comprising overlapping words from other speakers.

Preferably encoding sections of the pre-processed audio speech data into quantised audio representations comprises: encoding each section of pre-processed audio speech data into one of a fixed number of quantised audio representations, where the fixed number of quantised audio representations is preferably between 50 and 250,000, more preferably between 100 and 100,000. Providing a fixed number of quantised representation states means the quantised representations are inherently less identifiable. A number in this range provides enough states to represent the most important prosodic information but not so many that nuisance covariates (such as speaker characteristics or background noise) are represented. This provides representations which are expressive enough to represent e.g. 50 semantically meaningful pitches (24 quarter-tones across 2 octaves), 50 semantically meaningful pause lengths and 50 semantically meaningful word rhythms.

The computer-implemented method of any preceding claim wherein encoding sections of the pre-processed audio speech data into quantised audio representations comprises: inputting the pre-processed audio speech data into a prosody encoder, the prosody encoder comprising a machine learning model trained to map sections of the pre-processed audio data to corresponding quantised audio representations.

Preferably the machine learning model is trained using a contrastive, preferably self-supervised, signal. Preferably the machine learning model is trained using raw audio, preferably with no access to linguistic information.

Preferably the prosody encoder comprises: a first machine learning model trained to encode sections of the pre-processed audio data into corresponding audio representations; a second machine learning model trained to quantise each audio representations output from the first machine learning model into one of a fixed number of quantised audio representations. Preferably the first machine learning model is trained to encode sections of the pre-processed audio data into corresponding non-quantised audio representations. In this way, the first machine learning model has been trained to learn a first set of audio representations, where the first set of audio representations are not constrained to a fixed number of representations and the second machine learning model learns to map each first audio representations to a second set of quantised audio representations. Put another way, the second machine learning model performs vector quantisation on the representations learned by the first machine learning model. Preferably the first and second models are trained end to end, preferably using a self-supervised, e.g. masking, objective. The two stage encoding process allows for a first model to be configured to effectively extract audio features from the audio data and a second model optimised for encoding these into quantised representations.

Preferably the first machine learning model comprises a temporal convolutional neural network, preferably a temporal convolutional neural network with skip connections. Temporal convolutional neural networks are well suited to extracting audio features in raw audio data and have a large receptive field, configured to learn patterns in periodic signals naturally.

Preferably the second machine learning model is trained to perform vector quantisation on each non-quantised audio representation. Preferably the second machine learning model is trained to perform product quantisation on each non-quantised audio representation. Product quantisation allows for efficient quantisation of large vector spaces. Product quantisers also naturally disentangle their input so facilitate explainability of the model. In particular the product quantiser encourages disentanglement of the audio features into different components of prosody.

Preferably the pre-processed audio speech data is split into audio words, the audio words comprising variable length sections of the audio speech data, each containing one spoken word of the audio speech data, and the method further comprises padding each audio word to the same length before inputting into the prosody encoder. This adaptation allows for the use of a TCN with variable length audio as input, allowing the method to benefit from the advantages of using audio words as input into the encoder.

Preferably the method comprises inputting a sequence of quantised audio representations into a contextualisation model, the contextualisation model comprising a machine learning model trained to encode the quantised audio representations into corresponding contextualised audio representations which encode information relating to their context within the sequence. Contextualisation comprises encoding information on the relationship between one prosody representation and the surrounding prosody representations in a sequence of speech. Since the semantic meaning of prosody is contextual, contextualisation makes stronger prosody representations for predictions. Contextualization makes prosody representations with weaker cross-temporal interactions, which facilitates audio-linguistic representation learning. Preferably the contextualisation model comprises a transformer model.

Preferably the contextualisation model is trained using a masking objective, in particular a masked language modelling objective. In particular, the contextualisation model is trained by withholding a part of the input data and training the model to predict the withheld part of the input data. More specifically the contextualisation model is trained to predict a withheld part of the input data based on the context of the withheld part. More specifically one or more prosody representations in the input sequence are masked and the contextualisation model is trained to predict the masked representations based on the surrounding representations in the sequence.

Preferably the prosody encoder and the contextualisation model are trained end to end, wherein the prosody encoder and the contextualisation model may be preferred to as the encoder model. In particular preferably training of the encoder model (comprising the prosody encoder and contextualisation model) comprises inputting sections of the pre-processed audio speech data into the prosody encoder, masking one or more of the quantised prosody representations which are output by the prosody encoder and input into the contextualisation model and training the encoder model to predict the masked quantised prosody representations. Preferably the encoder model is trained using a contrastive objective wherein the model is provided with a number of possible quantised representations and is trained to predict which of the possible quantised representations is the correct quantised representation (which corresponds to the masked quantised representation). Preferably all of the possible quantised representations come from the same speaker. In this way the model is further encouraged to learn de-identified representations.

Preferably no text or language input is used during training. Preferably the model is trained to predict the output, in terms of quantised audio representations from audio input, encouraging the model to learn temporal patterns within the prosody signal.

Preferably the contextualisation model is configured to consider interactions between two quantised word representations in the sequence only up to a maximum number of separating words between the two quantised word representations, where the maximum number of separating words is within the range 10 to 50 words, preferably 20 to 40 words. Prosody has relatively short range correlations so the model is preferably configured to only consider interactions up to this number of words apart/Preferably the prosody encoder is trained using self-supervised learning, for example with a masking objective. Preferably self-supervised learning comprises learning on unlabelled data where the model creates labels using properties of the data. A masking objective, also referred to as a de-masking objective, involves training the model to withhold a part of the input data and training the model to predict the withheld part of the data. Preferably the encoder is trained using a masked language modelling objective. In particular preferably the encoder is trained to learn representations which allow the model to predict masked representations using the other representations in the input sequence.

Preferably the method further comprises training a probe model to map an audio representation of the encoder model (where the encoder model comprises the prosody encoder and contextualisation model) to an independently determined measure of a prosody feature. In this way, the prosodic information encoded in the audio representation of the model may be confirmed. This can be used in application of the representations for a speech analysis task to analyse the information encoded by the representations used to make a prediction. For example in the application to making a health condition prediction, probing can provide a quantifiable measure of the prosodic information encoded and predictive of a particular health condition, allowing more accurate or explainable diagnosis.

Preferably training of the probe model is independent to training of the encoder model. Preferably the audio representation is one or more of: a quantised audio representation (quantised prosody representation); a contextualised audio representations (contextualised prosody representation); a representation of the product quantiser. Preferably the probe model is trained to predict an audio feature representative of one of the subcomponents of prosody: pitch, rhythm, tempo and timbre. Preferably a probe model is trained for each audio feature for each subcomponent of prosody. For pitch, a probe model may be trained to predict the median pitch. For rhythm, probe models may be trained to predict median word intensity and number of syllables. For tempo, probe models may be trained to predict articulation rate (syllables per second), speech rate, average syllable duration, and word duration (including pre-silence). For timbre, probe models may be trained to predict the median formants F1, F2, F3 (shifted).

Preferably the probe model comprises a linear model, multi-layer perceptron, an attention-based model or a Bayesian neural network.

Preferably the probe model is configured to provide a quantifiable measure of the contribution of one or more components of prosody in the audio representation. Preferably the quantifiable measure is based on one or more of (1) the accuracy of the prediction of the prosody feature provided by the audio representation; (2) the size or complexity of the probe model required to provide a given prediction accuracy, and (3) the amount of input speech data needed to train the probe to achieve a given prediction accuracy.

Preferably the method comprises using information-theoretic probing using minimum description length.

Preferably the method further comprises combining the quantised audio representations with linguistic representations to form joint audio-linguistic representations. For example using a method described in European patent application number 20185364.5.

In a further aspect of the invention there is provided a computer-implemented method of training a machine learning model to map input audio speech data to de-identified audio representations of the input audio speech data, the method comprising: pre-processing the audio speech data to remove timbral information; inputting sections of the pre-processed audio data into a machine learning model and training the machine learning model using self-supervised learning to map the sections of the pre-processed audio data to corresponding audio representations.

Preferably training the machine model by self-supervised learning comprises training the model to predict withheld part of the input data. Preferably the model is trained using a masked language modelling objective.

Preferably the machine learning model comprises an encoder trained to map sections of the pre-processed audio data to quantised audio representations (or “tokens”) and a contextualisation model trained to map a sequence of the quantised audio representations to contextualised audio representations encoding information relating to their context within the sequence. Preferably the contextualisation model is a transformer.

Preferably the machine learning model is trained by masking one or more of the quantised audio representations and training the model to predict the masked quantised audio representations. Preferably a contrastive loss is provided wherein the model is provided with a limited number of quantised audio representations, wherein one is the masked quantised audio representation within the input sequence, and the model is trained to select the correct masked quantised audio representation. The encoder and the contextualisation model are preferably trained end to end so that the encoder learns to form predictive quantised audio representations during training. In this way, during training the model converges to form quantised audio representations which encode predictive prosodic information, allowing the model to predict surrounding prosodic information. Preferably the model is trained without access to any linguistic information contained within the audio speech data.

In a further aspect of the invention there is provided a computer-implemented method of performing a speech analysis task on input speech data, the method comprising: obtaining representations of the input speech data using the method of any preceding claim; inputting the representations into a task-specific machine learning model trained to perform a speech analysis task.

For example the task specific machine learning model may be one or more of:
a classifier trained to map the quantised audio representations to one or more categories, for example for classification of speech data as falling into a class associated with a particular health condition;
a regression model trained to provide a numerical value associated with a particular measure, such as a health condition, based on the input quantised audio representations, for example to give a value associated with a health condition severity score;
a sequence decoder which decodes the input quantised audio representations to an output sequence, for example to describe a change in an indicated disease overtime, where the model may be trained on labelled data using supervised training;
a clustering model which uses unlabelled data and is trained using unsupervised learning to sort the data into clusters with similar properties based on the input quantised audio representations, where this clustering of the data may be used to extract previously unknown health related trends in the input speech data.

Preferably the task-specific machine learning model is trained to provide a health condition prediction. Since prosodic information is particularly useful in diagnosing a wide range of health conditions the representations of the present invention are particularly well suited to speech analysis for monitoring or diagnosis of a health condition. Furthermore, the fact they are de-identified is particularly important in healthcare applications.

In some examples the health condition is related to the brain, for example a cognitive or neurodegenerative disease (example: Dementias, Alzheimer's Disease, Mild Cognitive Impairment, Vascular Dementia, Dementia with Lewy Bodies, Aphasias, Frontotemporal Dementias, Huntington's Disease); motor disorders (example: Parkinson's Disease, Progressive Supranuclear Palsy, Multiple System's Atrophy, Spinal Muscular Atrophy, Motor Neuron Disease, Multiple Sclerosis, Essential Tremor); affective disorders (example: Depression, Major Depressive Disorder, Treatment Resistant Depression, Hypomania, Bipolar Disorder, Anxiety, Schizophrenia and schizoaffective conditions, PTSD); neurobehavioural conditions (example: spectrum disorders, Attention-Deficit Hyperactivity Disorder, Obsessive Compulsive Disorder, Autism Spectrum Disorder, Anorexia, Bulimia), head injury or stroke (example: stroke, aphasic stroke, concussion, traumatic brain injury); pain (example: pain, quality of life)

Preferably the health condition is related to one or more of a cognitive or neurodegenerative disease, motor disorder, affective disorder, neurobehavioral condition, head injury or stroke. The methods according to the present invention are able to extract signals relating to the interrelation of language and speech which are particularly affected by changes in the brain and therefore the method is particularly optimised for detecting them.

In some examples the health condition is related to the respiratory system for example: SARS-CoV-2, Whooping cough, Asthma, COPD, Pneumonia, Wet/dry cough, Flu, Common cold, Lower respiratory Infections; Trachea, Bronchus, and Lung cancers; Tuberculosis).

In a further aspect of the invention there is provided a computer-implemented method of training a machine learning model for performing a speech analysis tasks, the method using training data comprising audio speech data, the method comprising: obtaining one or more linguistic representations that each encode a sub-word, word, or multiple word sequence, of the audio speech data; obtaining one or more audio representations that each encode audio content of a segment of the audio speech data using a method as described above or in the appended claims; combining the linguistic representations and audio representations into an input sequence comprising: linguistic representations of a sequence of one or more words or sub-words of the audio speech data; and audio representations of segments of the audio speech data, where the segments together contain the sequence of the one or more words or sub-words; the method further comprising: training a machine learning model using unsupervised learning to map the input sequence to a target output to learn combined audio-linguistic representations of the audio speech data for use in a speech analysis task.

By combining linguistic information associated with the words used in the speech data with non-linguistic information and training the machine learning model on the linguistic and non-linguistic representations jointly, the model can utilise complementary information and can be able to learn features associated with the interaction between language and audio components of speech (in addition to features relating solely to language and features relating solely to audio) which provide the model with discriminative abilities not present in existing techniques. In particular, by training the model on an input sequence of linguistic and audio representations the model learns joint audio-linguistic representations capturing information on the interrelation between the language used by a patient and the way it is spoken, including emotion, phonetic errors, deviations and hesitations.

Preferably a representation comprises a feature vector, i.e. a vector encoding important distinguishing attributes of the input data. The term embedding is used interchangeably with the term representation. Preferably a representation captures meaningful structure of the input by placing meaningfully similar inputs close together in the representation space. A representation can be learned and reused across models or at different stages of training.

In a further aspect of the invention there is provided a data structure comprising a quantised audio representation obtained from audio speech data by pre-processing the audio speech data to remove timbral information; and encoding a section of the pre-processed audio speech data into quantised audio representations.

For explainability purposes, we wish to measure how well a feature is represented in a given representation. We use the prequential (or online) approach to minimum description length (MDL) to quantify the regularity between representations and labels. Formally, MDL measures the number of bits required to transmit the labels given the representations. If a feature is highly extractable from a given representation, a model trained to detect said feature will converge quickly, resulting in a small MDL. Computing the MDL using the prequential approach requires sequential training and evaluation. We partition the train set into timesteps and train our probe such that at timestep to calculate the codelength as per the standard prequential method.

We further adapt this method to derive an information-theoretic definition of speech identifiability. Following the literature we consider the setup of a number of binary speaker verification trials but, instead of using equal error rate or log-likelihood-based metrics, we define the de-identification ratio of a set of trial representations with respect to enrolment representations as the inverse of the compression ratio of the theoretical minimum description length to transmit the data using a prequential approach. The rationale is that a shorter MDL means that the verification task is easier given the two representations. This improves upon prior work, which assumes a fixed model (usually a probabilistic LDA), by taking into account the effort required to perform verification as well as the performance on the task. Real attackers could have access to sophisticated models and arbitrary computational resources to compare speech representations, motivating this approach. Prior work performs verification on pairs of i-vectors; we likewise consider pairs of the same representation, but note that cross-representation comparisons ought to be included in more comprehensive studies, including raw audio and spectrogram as inputs. For simplicity, we mean-pool sequential representations over time but note that this could underestimate the identifiability of the sequence as a whole due to lost information.

In a further aspect of the invention there is provided a computer-implemented method of obtaining de-identified representations of audio speech data for use in a speech analysis task, the method comprising: encoding sections of the audio speech data into quantised audio representations by inputting sections of the audio data into a prosody encoder, the prosody encoder comprising a machine learning model trained to map sections of the audio data to corresponding quantised audio representations. Forcing the model to encode the audio data into a fixed number of quantised states means the model must be parsimonious with what it chooses to represent and therefore is encouraged to encode solely the prosodic information, which can be used to make a prediction during training, and not the speaker identifiable information which is not predictive. Preferably the encoder is trained to map the input audio data into one of a fixed number of quantised audio representations, where the fixed number of quantised audio representations is preferably between 100 and 100,000.

This aspect of the invention may include one or more of the above described features of the other aspects of the invention, or those defined in the appended claims, individually or in combination.

In a further aspect of the invention there is provided a computer-implemented method of obtaining de-identified representations of audio speech data for use in a speech analysis task, the method comprising: processing the audio speech data to remove speaker-characteristic information and encoding sections of the processed audio speech data into audio representations by inputting sections of the audio data into a prosody encoder, the prosody encoder comprising a machine learning model trained using unsupervised learning to map sections of the audio data to corresponding audio representations. Processing the audio data may comprise using a conditioning model to remove parts of the speaker-characteristic information, for example using an autoencoder conditioned one or more parts of the speech, for example the linguistic content or speaker-identifiable characteristics. Preferably the processing step comprises processing the audio data to remove timbral information. Preferably the prosody encoder is trained using self-supervised learning, for example using a masking objective. This aspect of the invention may have any of the above described preferable features of the other aspects of the invention, implemented alone or in combination.

### BRIEF DESCRIPTION OF THE DRAWINGS

Embodiments of the invention will now be described, by way of example only, with reference to the accompanying drawings, in which:

FIG. 1A schematically illustrates an overview of a method of extracting de-identified representations of audio speech data according to the present invention;

FIG. 1B schematically illustrates a preferable example of a method of extracting de-identified representations of audio speech data according to the present invention;

FIG. 2 schematically illustrates an overview of a possible encoder model architecture for use in the method of the present invention;

FIG. 3 schematically illustrates an overview of a possible prosody encoder I architecture for use in the method of the present invention

FIG. 4 schematically illustrates a possible prosody encoder block used in the model of FIG. 3.

### SPECIFIC DESCRIPTION

### Overview of Method

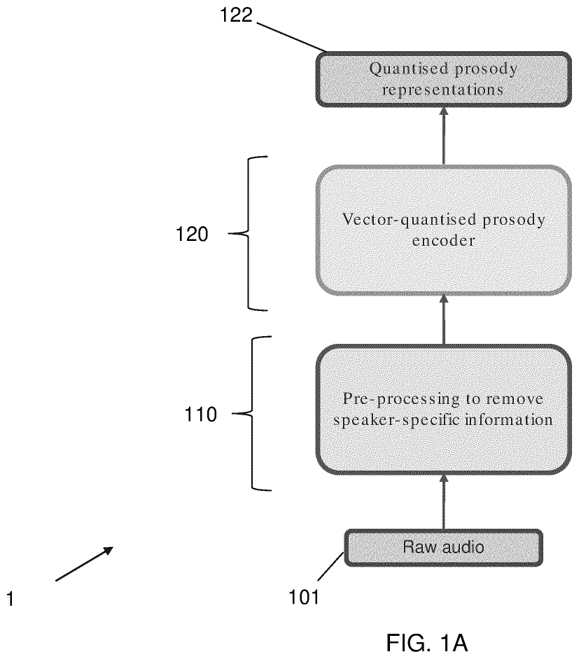

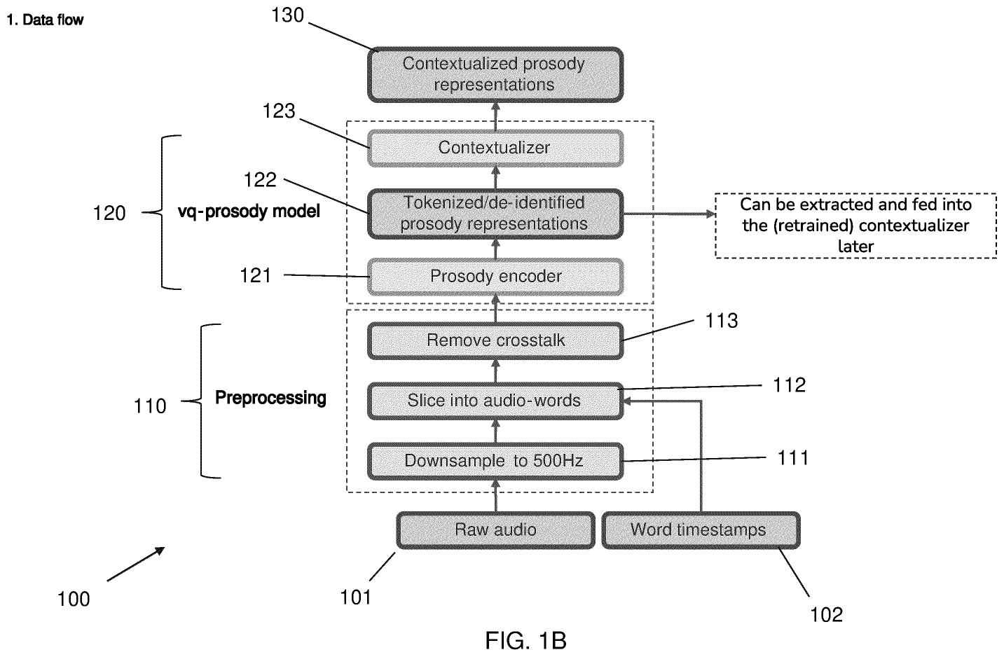

FIG. 1A schematically illustrates an overview of a computer-implemented method 1 for extracting de-identified prosody representations from input audio data 101 according to the present invention.

The method includes a data processing stage 110 and a prosody representation encoding stage 120. In the data processing stage 110 input speech data 101 is prepared, prior to being input into the machine learning model used in the encoding stage 120 to encode prosody representations of the input speech data 101.

In the pre-processing stage at least some speaker-specific information is removed from the raw audio speech signal 101. In particular, the audio speech data is processed to remove timbral information, where timbre refers to the voice characteristics of the speaker. Timbre comprises the formant frequencies, which are the resonant frequencies of the vocal tract. As described below, one or more pre-processing steps may be applied to remove all or part of the speaker characteristic component of the raw audio speech.

The pre-processed audio data is then input into the vector quantised prosody encoder 120, where the vector quantised prosody encoder comprises a machine learning model trained to map sections of input pre-processed audio speech data to quantised audio representations.

During training, the encoder model is trained to map sections of training data comprising input speech data, prepared using the data processing steps 110, to prosodic representations, as will be explained below. Then in use, the trained model 120 may be used to encode any input speech data, prepared using the data processing steps 110, into prosody representations 130 which are de-identified and may be used for downstream speech analysis tasks which require prosodic information to make a prediction, for example the monitoring and diagnosis of a health condition

The combination of pre-processing steps to remove speaker-characteristic portions of the audio signal together with the encoding in quantised audio representations together greatly reduce identifiability of the representations and encourage the learning of strong representations for downstream speech analysis. In particular, the method departs from established prior art techniques which use a subtractive method to encode prosody, for example by providing an encoder with the linguistic content of the input speech to define the remainder as prosody. Instead, by processing the raw data itself using a number of inductive biases to remove timbral information and training an encoder to encode the processed data into a fixed number of quantised prosody states, it has been found that the model can learn extremely strong prosody representations which are substantially de-identified from the speaker.

FIG. 1B illustrates a more specific example of the general method of FIG. 1A, where a number of possible pre-processing steps 110 and encoder model architectures 120 are illustrated.

The input to the method of this example is word-aligned raw audio of speech 101. In particular the input data is the raw audio 101 recording of speech with word timestamps 102 indicating the start/stop times for words but, unlike prior art methods, no linguistic information is provided with the audio data.

In the data processing stage 110, one or more pre-processing steps are applied to the input audio speech data. In the specific example of the figures the pre-processing stage comprises a downsampling step 111 in which the raw audio 101 is sampled at a frequency suitable to exclude spectral characteristics of speech (i.e. to remove timbral information from the data. This is followed by a slicing step 112 in which the audio data is split into audio words, i.e. sections of the audio data which each include a single word of the speech. If required, the pre-processing stage 110 includes a further step of removing crosstalk 113 in which any audio words including overlapping words from other speakers are excluded.

The subsequent encoding stage 120 comprises encoding the audio words of the processed input audio data into vector quantised prosody representations (also referred to as prosody “tokens”). First individual audio words are fed into a prosody encoder 122 trained to encode an audio word into a corresponding vector-quantised prosody representation (a prosody token) 122. A sequence of prosody tokens is then fed into a contextualizer model 123 trained to map a prosody token 122 within a sequence of prosody tokens to a vector-quantised contextualised prosody representations 130 (contextualised prosody representations), which encodes information about its relationship with surrounding tokens within the sequence.

Both the individual prosody tokens 122 and the contextualised prosody tokens 130 are deidentified from the speaker and can be used in downstream tasks, such as encoding speech for the monitoring and diagnosis of a health condition or expressive text-to-speech systems and spoken language understanding.

Below, various possible features of the data processing stage 110 and the encoding stage 120 are explained in more detail to illustrate the rationale for their inclusion and how they contribute to providing strong de-identified prosody representations.

Data Processing Stage

The data processing stage 110 can include one or more steps for pre-processing the audio speech data 101 to (1) increase the amount of prosodic information encoded in the prosodic representations learned by the model and/or (2) enhance the de-identification of the prosody representations. Generally, the data processing can include one or more signal processing steps and/or the application of a machine learning model trained to remove timbral information, for example using an encoder conditioned on a component of the input speech.

Input Data

A first consideration is the type of input speech data to use in the method. Preferably the method according to the present invention uses raw audio as the input. Unlike many prior art methods in which processed audio, such as a spectrogram, is used as input, the inventors have determined that inputting raw audio into the model provides significant improvements in the models ability to encode prosodic information that occurs at multiple time-scales in input speech data.

Unlike spectrograms, raw audio includes all information within the speech and does not introduce bias or lose any information by first converting the audio into a format intended to be more readily processable by the model. This gives the network the flexibility to learn whatever it chooses and encode richer information within the representations.

In previous models using raw audio as the input is generally not computationally feasible, but combined with other aspects of the method according to the present invention it is possible benefit from the richer information within the raw audio input while still being computationally feasible, as explained below.

Another notable feature of the present method for obtaining prosodic representations is that it only requires audio data as the input and as the target when training the model. The model therefore learns prosody representations without having to use words/phonemes as input data by relying on predicting temporal patterns within the prosodic information alone. Prosody in speech has predictable temporal patterns and the inventors have determined that this can be used to train the model to learn strong prosodic representations, without requiring linguistic information to be fed into the model.

As described below, one particularly preferable approach is using a contrastive, self-supervised training method, where only raw audio is used as input and targets.

Downsampling the Audio Input

A first important step is that the inventors have realised that significantly downsampling the input audio data ensures that spectral characteristics of the speech (timbre) are excluded whilst preserving other prosodic information. This approach is built on the fact that timbral information (characteristic of the speaker) is found at higher frequencies than other, non-identifying parts of prosody. Sampling at a suitable low enough frequency can therefore ensure the network learns about prosody, not phonetics.

In the example of the figures the raw audio is sampled at 500 Hz. The rationale for this figure is that applying the Nyquist theorem on the highest typical female fundamental frequency (F0=255 Hz) is roughly 500 Hz. Therefore this sampling rate retains pitch and rhythm information found in the F0 contour, but removes spectral information, such as the formants, that characterizes the speaker.

In this way, a majority of the identifiable spectral information in a voice is already removed by this initial processing step. This approach departs significantly from prior art techniques, where the focus on retaining as much information in the raw speech signal as possible would appear to contradict such a low frequency sampling of the input data. However, this kind of downsampling in fact preserves the important prosodic information required for many speech analysis tasks, while excluding phonetic information characteristic of the speaker—providing both stronger prosodic representations and reducing identifiability.

A significant technical advantage associated with this degree of aggressive downsampling is that it makes the input sequence for a word (which may be around 1 s in length) a computationally feasible length. In particular, as described above, the current method preferably uses raw audio which is too computationally intensive to process with currently available systems at conventional sampling frequencies. By downsampling at much lower frequencies (e.g. compared to the ˜16 kHz sampling typically used to standardise data for input to a neural network) the prosodic information is maintained in signal whilst allowing the network full flexibility to encode the information due to the use of raw audio data.

Aligning the Input Audio by Words

A further important pre-processing step applied to the input audio speech data is to align the input audio words. This may be applied independently of, or in addition to, the other pre-processing steps described. This word alignment step involves aligning the input audio by words so that each prosodic representation corresponds to a single word. In particular the input audio is split into segments of periods of audio data, each corresponding to a word. These sections may be of variable length with a maximum duration preferably between 0.5 and 3 seconds in length, preferably around 2 seconds. This may be achieved by using word timestamps which indicate the divisions between words in the audio speech data. Alternatively word start and end timestamps may be used which indicate the bounds of the audio data around a particular word. These sections of audio data corresponding to a single word (referred to herein as “audio words”) are then input into the prosody encoder so that the encoder learns one prosodic representation for each word.

The rationale behind this is that prosody is strongly temporally associated with words and semantically meaningful prosody states are naturally discretized on a per-word basis. The inventors have therefore determined that using a variable audio segment length, corresponding to the word length in the speech, enables the model to learn stronger prosody representations. This significantly departs from most prior art methods in which the input audio is split into fixed length audio segments, generally of the order of ˜10 ms rather than ˜1 s as in the present invention, to be input into a model for encoding into representations.

Using long sequences of audio segments on the word level is made more computationally feasible in part due to the downsampling used.

Including Time Between Words

A preferable addition to the word-level alignment of the audio is to include a period of silence (i.e. non speech audio) in each audio word. The time between words includes significant prosodic information, such as hesitations, speech rate, stuttering etc. The speech rate and temporal variation in particular is important information to represent in the prosody representations for downstream speech processing tasks. The time preceding the spoken word is more relevant to the word than the time following it, it being more directly linked with the cognitive processes of the speaker. Therefore preferably the method involves including a period of preceding silence (non-speech audio) in each audio word. The period is preferably up to 1 second or up to 2 seconds in length.

Preparing sections of audio speech data including a word and period of preceding silence as input to the encoder ensures that the representations learned by the model during training encode a greater amount of prosodic information, in particular information about absolute and/or relative speech.

Normalising Baseline Pitch

A further data processing step, which may be used individually or in combination with one or more of the above steps, to improve strength of the prosodic representations learned by the model is to normalise the baseline pitch.

The baseline pitch of speech is a characteristic feature of speech and therefore can be used to identify a speaker. Furthermore, tt is not information which is useful for understanding spoken language in order to make predictions based on the speech. However the variation from the baseline pitch in speech data is an important element of prosody which should be encoded in the prosodic representations learned by the model.

The inventors have therefore determined that improved results can be obtained by pitch-shifting the input data to a predetermined frequency so that the median pitch of voiced segments is the same across speakers. This firstly increases the efficiency of the representations learned by the model as it restricts the model from encoding unnecessary information about the baseline pitch of an element of speech within the representations. Secondly, and particularly importantly, the baseline pitch is indicative of a particular speaker, so removing the baseline pitch information makes the representations less identifiable.

A further technical advantage is that reducing the range of pitches in the dataset aids quantisation of the representations, enabling a smaller codebook. It also stabilises training and speeds up conversion.

Prosody Encoder Model

After the input speech data has been processed, the word length sections of audio data (referred to as “audio words”) are input into a machine learning model trained to map the audio words to quantized prosody representations (referred to as prosody tokens).

### Overview of Example Encoder Model Architecture

The prosody encoder model may be any model suitable for encoding the pre-processed sections of audio data into quantised audio representations. The prosody encoder preferably includes a machine learning model, trained to map sections of processed audio data to corresponding quantised audio representations of the sections of audio data.

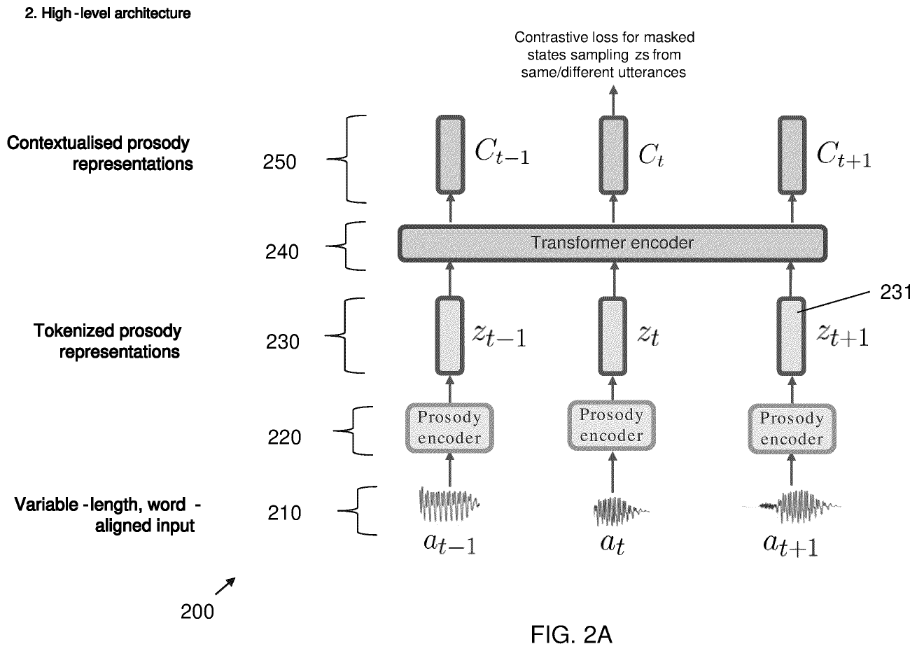

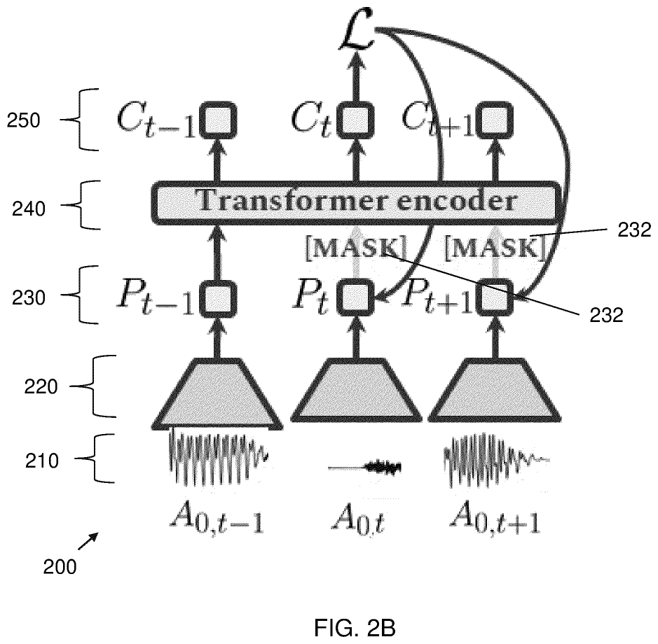

FIG. 2 schematically illustrates a high-level view of an example of a possible prosody encoder model 200.

The input 210 to the model is sections of the pre-processed audio data. Preferably this comprises variable length, word-aligned audio, i.e. sections of the processed audio data which each include one spoken word. These sections of processed data are referred to as “audio words”.

The first stage of the model is the prosody encoder 220. This is a model, or series of models, configured to take one audio word as input and encode this single word as a corresponding quantised audio representation encoding the prosodic information of the audio word. Prosodic information is effectively encoded due to the pre-processing to remove speaker-identifying information from the raw audio input, in particular timbre, and due to various features of the model, described in more detail below.

The output of the prosody encoder stage 220 is therefore a sequence of quantised prosody representations 230, each encoding the prosodic information of one spoken word within the input speech and therefore together in sequence encoding the prosodic information of a length of audio data.

The prosody encoder 220 may have several possible different structures. As described below, in one example the prosody encoder comprises a first stage configured to encode each input audio word as a non-quantised audio representation and a second stage configured to quantise each non-quantised audio representations into one of a fixed number of quantised prosodic states (quantised prosody representations or prosody tokens). Further possible implementation details of the prosody encoder are set out below.

The sequence of prosody tokens 230 is then fed into a contexualiser model 240 to encode the quantised prosody representations into contexualised prosody representations. The contextualisation model 240 is preferably a sequence-to-sequence machine learning model configured to encode contextual information of a particular prosody token 231 into a new representation. The model is configured to encode information about the relationships between a quantised prosody representation 231 and the surrounding quantised representations within the sequence 230—commonly referred to as “context”. The contextualisation model 240 is preferably an attention based model, in particular a transformer encoder.

The output of the contextualisation model 240 is a sequence of contextualised prosody representations 250, each encoding the prosodic information of a particular audio word in the sequence and its relationship to the surrounding prosodic information in the sequence.

Both the tokenized prosody representations 230 or the contextualized prosody representations 250 can be used for downstream tasks, like expressive text-to-speech systems, spoken language understanding and speech analysis for the monitoring and diagnosis of a health condition. Both sets of representations encode just the prosodic information of the speech and are substantially de-identified so may be used where anonymising of user data is required.

Overview of Model Training

FIG. 2B schematically illustrates a method of training an encoder model of FIG. 2A for use in the method according to the present invention.

Firstly the pre-processing is carried out on a training data set comprising raw audio speech data. The pre-processed raw audio 210 is fed into the prosody encoder 220, which produces one set of prosody tokens (P_i) 230 for each audio-word 210. In the illustrated example there are 3 tokens for each audio-word 210 but it can be 1 or more. At this stage, the model is completely non-contextual—each representation has only ever seen the audio for its own audio-word and not any information from the surrounding parts of the audio data. As described above the mode then comprises a contexulisation encoder 240, preferably a transformer, configured to encode the prosody tokens into contextualised representations 250.

The training process used is a form of self-supervised learning in which the model is trained to predict masked tokens from the surrounding context. This is a similar approach to that used in masked language models (see for example “BERT: Pretraining of Deep Bidirectional Transformers for Language Understanding”, Devlin et al. arXiv:1810.04805) but in this case the model uses solely audio, prosodic information and instead of training the model to predict the masked token a contrastive training approach is used in which the model is trained to predict the correct token from a number of different tokens.

In more detail, one or more tokens 230 output by the prosody encoder 220 are randomly masked 232, the model is given a number, for example 10, possible tokens and the model is then trained to predict the correct one from the group of possible tokens (i.e. which token corresponds to the token that has been masked). The other 9 tokens are masked states from other masked audio-words. One preferable feature of the training process is that the other tokens (the negatives) are selected from the same speaker. In this way the model is not encouraged to encode information that helps separates speakers and therefore further aids de-identification of the representations.

The network 200 is trained end to end so the prosody encoder 220 is trained together with the transformer encoder 240.

Preferably the model is trained using a contrastive loss so that it can robustly converge to meaningful prosody representations when being trained end-to-end. Once trained, input speech data can be fed into the model and either or both of the contextual representations (post-Transformer) or the pre-Transformer non-contextualized representations (or from any layer inside the Transformer) can be used for downstream speech processing tasks.

Advantageous Features of the Encoder Model

As with the pre-processing stage, there are a number of steps that may be taken, either alone or in combination, in selecting and implementing the model architecture to improve both (1) the amount of useful prosodic information encoded in the representations for use in a downstream speech analysis task and (2) de-identification of the prosodic representations.

The below features of the model may be implemented individually or in combination to provide the described advantages.

Use of Vector-Quantised Representations

The model is preferably structured to learn vector-quantised prosodic representations, or “tokens”, encoding the segments of input audio data. In particular, the model preferably uses a fixed number of quantised prosody states and each audio word is mapped to one of these states. The use of tokenised prosodic representations provides a number of crucial advantages.

It firstly encourages the model to learn parsimonious representations, that is, representations which efficiently encode the most important prosodic information. The most important information for making predictions during self-supervised learning is prosodic so the model encourages learning of representations which encode this information and avoid encoding nuisance covariates, particularly information relating to the speaker identity, such as age, gender etc. In this way, using vector-quantised representations also improves de-identification as the limited number of prosodic states mean that only prosodic information is encoded and not information relating to identifiable characteristics of the speaker.

The method preferably uses 50 to 250k quantised prosody states, more preferably 100 to 100k states. Particularly preferable examples use around 125,000 quantised prosody states. Limiting the number of quantised prosody states in this way provides enough states to represent the most interesting prosody information but not so many that nuisance covariates, such as background noise, speaker characteristics etc, get represented. Limiting the number of states increases deidentifiability.

As an illustrative example, 125,000 quantised prosody states is expressive enough to represent e.g. 50 semantically meaningful pitches (24 quarter-tones across 2 octaves), 50 semantically meaningful pause lengths and 50 semantically meaningful word rhythms.

The number of states can be significantly further reduced to further increase deidentifiability, for example using between 1000 and 10k states.

Particularly when combining the use of a limited number of states and working on a time scale of ˜0.5 s per representation (i.e. for one word), rather than ˜20 ms as is standard with normal speech representations, the de-identification is greatly enhanced. The fact that longer periods of speech are forced into a relatively small number of prosodic states, meaning the likelihood of the original speaker being identified based on these prosodic states encoding their speech is extremely low.

Use of a Temporal Convolutional Network to Extract Audio Features

Since the prosody is encoded in the input audio signal, it is beneficial to use a model architecture well suited to learning patterns in raw audio in order to extract the audio features to then form initial non-quantised prosody representations which are then quantised. The method preferably implements a temporal convolutional network (TCN) to extract the audio features from the individual audio words. A TCN (see for example “An Empirical Evaluation of Generic Convolutional and Recurrent Networks for Sequence Modeling”, S. Bal et al, arXiv:1803.01271v2 19 Apr. 2018) is a particular arrangement of convolutional layers with ‘dilating’ layers that gives it an exponentially increasing receptive field size as the number of layers increases. It acts like a recurrent neural network (RNN) but can capture information from much longer sequences.

The use of a TCN or similar model permits a large receptive field (for example 1,280 frames) and the model learns patterns in periodic signals naturally. Although TCNs are used in certain preferable embodiments of the invention, other models may be used to extract features from the audio word segments in order to form initial non-quantised representations.

Use of a Contextual Encoder

The semantic meaning of prosody is contextual, that is the meaning associated with the prosody of a spoken word is related to prosody of the preceding and following speech. Therefore to encode the most prosodic information of an element of speech it is necessary to encode contextual information, taking into account the prosodic information of the surrounding speech. Contextualisation makes stronger prosody representations for making predictions in downstream speech analysis tasks, for example in making predictions relating to a health condition of the speaker. Furthermore contextualization makes prosody representations with weaker cross-temporal interactions, which helps with audio-linguistic representation learning.

Therefore preferably the model comprises a contextual encoder arranged to encode contextual prosodic information into output contextualised prosody representations. A suitable encoder model may be based on the Transformer architecture (see “Attention is all you need”, Vaswani 2017, arXiv:1706.03762v5).

Prosody has relatively short-range interactions so the model may be configured to consider temporal interactions only up to 32 words apart. Prosody is strongly associated with sentences, so when feeding the input into the contextual encoder it is preferable to chop the audio-words into sentences rather than arbitrarily to preserve this structure.

Only Using Audio as the Input and Target Output During Training

Prosody as predictable temporal patterns and the inventors have determined that predicting prosodic states based on prosody alone requires similar prosody representations as predicting prosodic states using words. The model is therefore preferably structured to only take audio as input and is trained to predict masked audio words using the prosodic representations. This departs from prior art methods in which generally linguistic information is fed to the model to encourage the model to learn prosodic representations. In this way the model learns prosody representations without having to use words/phonemes as input data by relying on predicting temporal patterns requiring strong representations of similar information.

### Specific Example of a Prosody Encoder

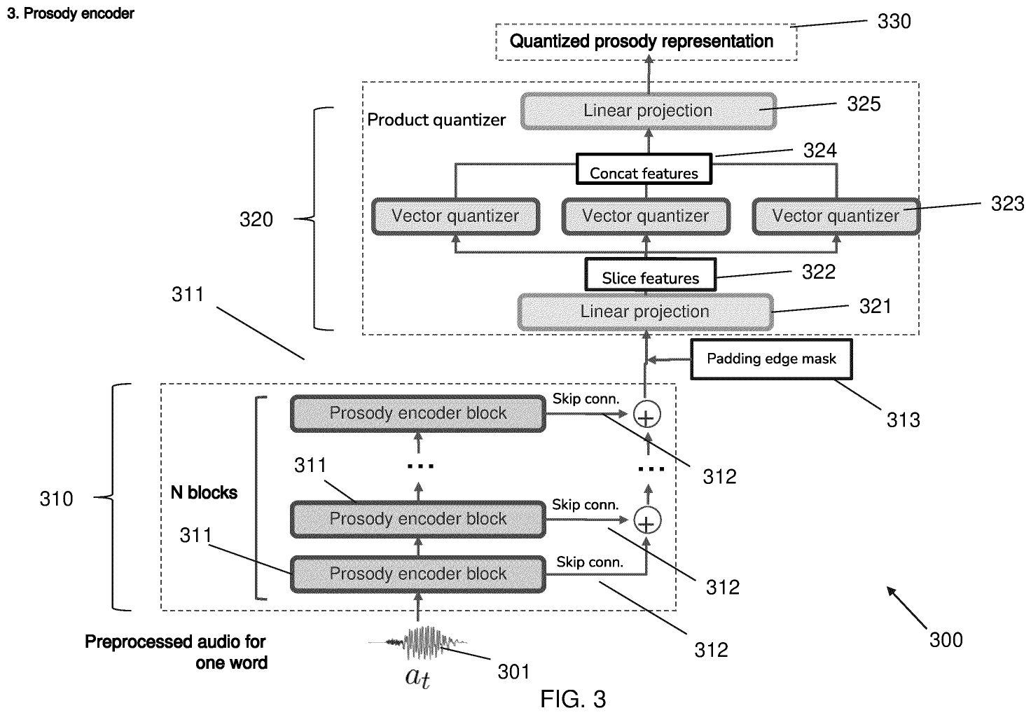

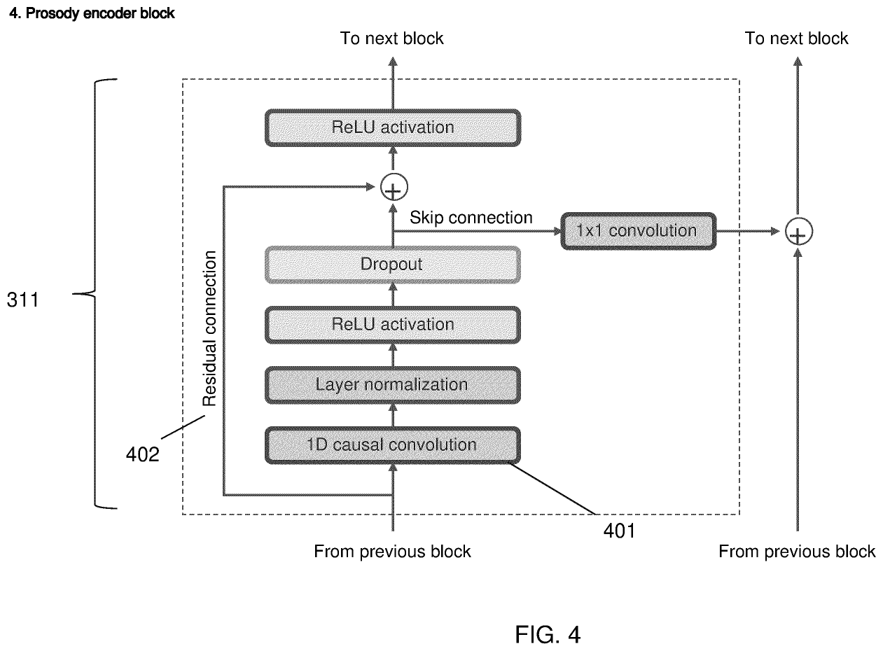

FIGS. 3 and 4 schematically illustrate one possible example of a prosody encoder 300 suitable for use in the current invention. As described above, the prosody encoder is any model configured to take audio words as input and encode these into quantised prosody representations. However, a preferable architecture is illustrated in FIGS. 3 and 4.

In this example of the invention, the input to the prosody encoder is an audio word 301, i.e. a section of the audio speech data including one spoken word, preferably pre-processed to remove timbral information. This may be achieved by a number of pre-processing techniques, including downsampling the data to ˜500 Hz and normalising the baseline pitch.

Non-Quantised Prosody Encoder

As shown in FIG. 3, the raw audio for one audio word is input into a series of prosody encoder blocks 311, together called a temporal convolutional network (TCN) 310 (see for example Oord, A. et al. Wavenet: A generative model for raw audio. arXiv preprint arXiv:1609.03499, 2016). Each block 311 has the same structure (an example of which is shown in FIG. 4) and uses dilated convolutions to identify patterns at various timescales. The first layers are configured to learn simpler patterns occurring at a shorter time scale, whereas deeper layers can learn more complex patterns with longer term dependencies.

More specifically the TCN comprises a stacked set of identical layers/blocks, where the input to layer 1 is the raw audio, then the input to layer 2 is the output from layer 1, and the input to layer 3 is the output of layer 2, and so on. In this way each layer can build more complex abstractions of the input but critically each layer can also learn patterns on larger timescales than the previous layer, which means the model can take a long sequence of e.g. raw audio and extract information from it.

Preferably the temporal convolutional network (TCN) comprises of a stack of causal dilated 1D convolutions with residual connections, which we adapt with skip connections. The strides, number of layers and kernel sizes are chosen such that the receptive field of the TCN spans the maximum sequence length of one audio word.

The output of the last later could be taken as the TCN output to be fed into the product quantizer but preferably information from every layer is pulled out of the using skip connections (as described in WAVENET: A GENERATIVE MODEL FOR RAW AUDIO, van den Oort et al, arXiv:1609.03499v2 19 Sep. 2016) and the model uses the summed output of the skip connections as the initial non-quantised prosody representations. The use of skip connections, pulling out information from every layer, allows information from different timescales to be assembled more easily.

FIG. 4 illustrates a possible internal structure of a prosody encoder block 311 of the TCN 310. The block has a 1D convolutional layer that can find temporal patterns. This is configured so that the number of timesteps the model sees with each successive layer increases exponentially—referred to as dilated causal convolutions, which is typical of TCNs.

A further important feature of each block 311 of the TCN 310 is the residual connections 402. The residual connections provide a path through which information can skip a layer. This allows the model to preserve information more naturally from previous layers if required. The other elements of the block, the layer normalization, ReLU activation and dropout are the standard elements employed to normalize and make the training more robust.

The TCN 310 is preferably designed such that its receptive field corresponds to about 1 s of speech, to cover most audio-words. The final element in the output TCN sequence is able to encode information about everything within its receptive field.

In preferable examples of the invention using variable length audio words 301, these are batched together by padding them all to the same length, so the summed skip connection output undergo edge masking 131 to mask the padded section and extract the final timestep in each case.

Product Quantizer

As shown in FIG. 3, the features extracted using the TCN 310 are used to form a non-quantised audio representation of the input audio word 301. This is fed then into a product quantizer 320 which is configured to take a vector as input and output a quantised vector (i.e. a token). More specifically the product quantizer is trained to learn a linear mapping into a new space where its features can be split up into N parts and quantized independently, before being recombined. This creates a set of SAN possible states, where S is the number of states allowed by each individual quantizer. The resulting N tokens are a de-identified, quantized/tokenized representation of prosody.

The product quantiser may have a similar structure to that described in “wav2vec 2.0: A Framework for Self-Supervised Learning of Speech Representations”, arXiv:2006.11477. Product quantisation is an extension of vector quantization that allows much larger spaces to be quantised efficiently, by decomposing a space into multiple orthogonal subspaces and vector quantize each one independently, before uniting the tokens.

In more detail and with reference to FIG. 3, firstly a linear projection 321 is performed to map the non-quantised representation to a new feature space. Its features are then split into N parts (in this case 3), with each set of features quantised independently with a vector quantiser 323 into a set of S states.

The number of quantised states is deliberately restricted while learning the vector-quantized representations to promote representations to be parsimonious and avoid “hiding” nuisance covariates in small detail. This increases robustness, reliability, and generalisability of the method, based on the fact that the most important information for making predictions during the self-supervised training of the model is the prosodic information so this will be preferentially encoded in the representations. The smaller codebook is made possible partly due to the downsampling and normalising of pitch that are applied during preprocessing of the data.

A further advantage of using product quantisation to quantise the non-quantised prosodic representations is the possibility of disentangling the representation space into more readily understandable factors, increasing explainability of downstream speech analysis tasks. The inventors have determined that product quantisers will naturally disentangle their input, so each quantiser 323 in FIG. 3 is encouraged to learn something different. In particular, by restricting the number of factors N used in the product quantisation to N=3, it is possible to train the model to disentangle the components of I prosody. In this way, the method can provide quantised prosodic representations which are readily interpretable and allow an analysis of the specific components of prosody that are involved, for example in analysing changes in prosody in the application of speech analysis to diagnosis Alzheimer's.

Returning to FIG. 3, the features are then concatenated 324 with a linear projection 325 then performed to provide the output quantised representations of prosody having a total of S″ states. The three (in this example) quantizers are each def ⅓ of the features so the linear projection layer 321, 325 before and after are configured to encourage the network to disentangle the features before they are sliced and send to the vector quantisers 323, then help recombine them.

The product quantizer is configured to split the features at stage 322 into meaningful features spaces for example pitch, pause length and rhythm. The total number of quantised prosody states may be limited, for example to e.g. 50 semantically meaningful pitches (24 quarter-tones across 2 octaves), 50 semantically meaningful pause lengths and 50 semantically meaningful word rhythms (i.e. the number of states allowed by each vector quantiser 323 S may be 50, with 3 different feature spaces) to give 125,000 possible quantised prosody states.

In this way, each audio word 301 is encoded into one of the possible quantised prosody representations 330.

Applications of the De-Identified Prosody Representations

High quality data representations encoding non-linguistic information within speech are required for a large number of applications. Speech data can be encoded within the prosody representations using the present method and then used for a wide range of downstream speech analysis tasks, for example using a machine learning model trained to perform a particular task on input speech data encoded in the prosody representations of the present invention, such as classification, regression or clustering tasks. The representations can improve any machine learning model tasked with understanding speech data and producing expressive text-to-speech.

Many of these fields, and particularly speech analysis for health applications, require that these data representations are sufficiently de-identified to protect user privacy and meet GDPR/HIPAA requirements. Certain limited examples include:
Automatic speech recognition.
Diarisation (separating speakers during automatic speech recognition).
Lie detection.
Sarcasm detection.
Personality prediction.
Sentence acceptability.
Sentiment analysis.
Paraphrasing/sentence similarity.
Natural language inference.
Coreference resolution.
Sentence completion.
Word sense disambiguation.
Question answering.
Machine translation.
Understanding intent.
Conversational agents such as chatbots.
Text-to-speech
Speech generation/synthesis.
Style transfer/voice conversion.
Predicting states such as fatigue, attention, and effort.

A particularly important application of the quantised audio representations is for speech analysis for monitoring or diagnosis of a health condition, where changes in the non-linguistic content of speech are associated with a wide range of health conditions.

There are a huge number of health conditions which leave signals within speech which can be identified by encoding patient speech using the data structures extracted using the methods of the present invention. A few limited examples include: where the health condition is related to the brain, e.g. a cognitive or neurodegenerative disease (example: Dementias, Alzheimer's Disease, Mild Cognitive Impairment, Vascular Dementia, Dementia with Lewy Bodies, Aphasias, Frontotemporal Dementias, Huntington's Disease); motor disorders (example: Parkinson's Disease, Progressive Supranuclear Palsy, Multiple System's Atrophy, Spinal Muscular Atrophy, Motor Neuron Disease, Multiple Sclerosis, Essential Tremor); affective disorders (example: Depression, Major Depressive Disorder, Treatment Resistant Depression, Hypomania, Bipolar Disorder, Anxiety, Schizophrenia and schizoaffective conditions, PTSD); neurobehavioural conditions (example: spectrum disorders, Attention-Deficit Hyperactivity Disorder, Obsessive Compulsive Disorder, Autism Spectrum Disorder, Anorexia, Bulimia), head injury or stroke (example: stroke, aphasic stroke, concussion, traumatic brain injury); pain (example: pain, quality of life).

Further limited examples include where the health condition is related to the respiratory system (example: SARS-CoV-2, Whooping cough, Asthma, COPD, Pneumonia, Wet/dry cough, Flu, Common cold, Lower respiratory Infections; Trachea, Bronchus, and Lung cancers; Tuberculosis).

The methods described herein can also be applied to cases where there are multiple different health conditions or symptoms of different health conditions or where the health conditions are not yet known.

Probing of the De-Identified Prosody Representations

In a further optional extension of the method the prosodic representations may be probed to confirm that they are encoding prosodic information and to understand the type of prosodic information encoded. This technique is useful in a wide range of applications, for example when using the representations to make a health condition prediction, probing the information that is encoded can allow a clinician to understand the components of prosody that are most affected by a health condition, allowing the system and clinician to achieve a more specific and accurate diagnosis of a health condition.

The method involves training a probe, comprising a machine learning model, independently to the training of the prosody encoder model, to map a representation of the input speech data to an independently determined measure of prosody or a measure of one of the components of prosody. By examining the success of the model in predicting a component of prosody it can be determined to what extent the prosodic representations encode information in speech related to that component. Furthermore, and importantly, probing can provide a quantifiable measure of the success of predicting a particular measure of prosody. Therefore when the method is applied in a technical application, this quantifiable probing technique, can provide a quantified measure of the prosodic representations' success in encoding the relevant prosodic property, which can be provided as an output to a user.

Of particular relevance for the present invention is confirming that the prosodic representations encode each of the required components of prosody, other than the speaker identifying characteristics—timbre in particular. Therefore the method may further comprise training a probe model to predict audio features representative of the subcomponents of prosody: pitch, rhythm, tempo and timbre.

For pitch a probe model may be trained to predict the median pitch. For rhythm probe models may be trained to predict median word intensity and number of syllables. For tempo, probe models may be trained to predict articulation rate (syllables per second), speech rate, average syllable duration, and word duration (including pre-silence). For timbre, probe models may be trained to predict the median formants F1, F2, F3 (shifted).

To quantify how well the trained representations encode the prosodic information, the method may use the accuracy of the probe or more preferably it may employ information-theoretic probing with minimum description length (as described in “Information-Theoretic Probing with Minimum Description Length”, Proceedings of the 2020 Conference on Empirical Methods in Natural Language Processing, pages 183-196, Nov. 16-20, 2020). This technique provides an objective measure of how well information is encoded in the quantised audio representations for each of the audio features representative of each subcomponent of prosody.

In terms of a code, the ability of a probe to achieve good quality using a small amount of data or using a small probe architecture reflect the same property: the strength of the regularity in the data.

The probe models may be applied to both the quantised prosodic representations output from the product quantiser and the contextualised prosodic representations output from the contextualisation model, to provide an output to a user to inform on the information that is being encoded. The probe models may also be applied to the components of the product quantizer, i.e. the three components of the vector quantizers 323 shown in FIG. 3. The application of the latter has shown that the product quantizer described has the ability to naturally disentangle the information into the three non-timbral components of prosody.

The probe models comprise a machine learning model, preferably a simple classifier or regression model, trained separately to the encoder models to map one or more audio representations provided by the model to a measure of prosody. The probe preferably comprises a linear model, multi-layer perceptron, an attention-based model or a Bayesian neural network and is preferably simple such that it does not internally learn to do the task in a sophisticated way.

### Example of Specific Implementation of Model and Testing

The following sets out one specific non-limiting implementation of the method according to the present invention, including specific choices for each of the individually separable features described above, covering the model architecture and training method and details of testing of the model to confirm de-identification of the quantised prosodic representations.

Model Architecture

In one example, the model comprises two parts: a prosody encoder and a Transformer encoder (see FIGS. 2A and 2B). The prosody encoder maps variable-length raw audio corresponding to a single audio-word, to a fixed-length quantized vector (Pt in FIG. 2B). The sequence of latent prosody representations Pt is fed to a Transformer to produce contextualized prosody representations Ct that can capture information from the entire sequence, unlike the audio-word-level Pt representations. Prosody has predictable temporal patterns, occurring at frequencies lower than 250 Hz that can be learned directly from the acoustic signal. A contrastive, self-supervised signal is used to train the model, (similar to Baevski, A., wav2vec 2.0: A framework for self-supervised learning of speech representations. arXiv preprint arXiv:2006.11477, 2020), where only raw audio is used as the input and target. Unlike subtractive approaches to representing prosody the model does not rely on lexical inputs. Instead, it only has access to the downsampled raw audio signal and word-level timestamps.

Temporal Convolutional Network

The first module of the prosody encoder is a temporal convolutional network (TCN) comprising of a stack of causal dilated 1D convolutions with residual connections, which we adapt with skip connections. The strides, number of layers and kernel sizes are chosen such that the receptive field of the TCN spans the maximum sequence length of one audio word. Skip-connections are used (see Oord, A. v. et al, Wavenet: A generative model for raw audio. arXiv preprint arXiv:1609.03499, 2016) rather than the output of the final layer to allow the network to more easily capture features with different time-resolutions. The skip connections are passed through a 1×1 convolution to relax the constraint that the convolved data passing to the next TCN layer (after being summed with the residual) must be identical to the output skip matrix. To reduce across the temporal (frame) dimension, the skip matrix is max-pooled, which the inventors empirically found led to more robust training than selecting the final non-padded element in the skip matrix for each element in the batch. The exponentially increasing receptive field of our TCN to capture the longer range dependencies that encode prosodic information.

In this specific example the TCN comprises 9 layers, each with 30 filters, a stride of 1 and a kernel size of 2. We use exponentially increasing dilations of size 1, 2, 4, 8, 16, 32, 64, 128, 256 to yield a receptive field size of 512 frames. The 1×1 convolution similarly has 30 filters. The dropout probability is 10%.

Product Quantizer

The max-pooled output of the TCN is passed to a product quantizer, comprising constituent vector quantizers is inspired by VQ-VAE-2 (see Razavi, A., Oord, A. v. d., and Vinyals, O. Generating diverse high-fidelity images with vq-vae-2. arXiv preprint arXiv:1906.00446, 2019) but adapted for product quantization. The product quantizer itself is similar to wav2vec 2.0, wherein the input data undergoes an affine transformation before having its features sliced into M equal parts, all of which are passed to a vector quantizer. Following quantization, the quantized output vectors are concatenated and undergo a final affine transformation. Following (Razavi et al., 2019), each constituent vector quantizer learns a nonlinear mapping from its input space S to a vector E(s), which is replaced with the nearest prototype vector in the codebook eₖ, k ξ1, . . . , K:Quantize (E (s))=eₖ. This mapping is learnt via backpropagation using the straight-through gradient estimator. Using multiple vector quantizers is not equivalent to using one with a larger capacity; the inclusion of affine transformations before and after the vector quantization gives the network some capacity to map the input data into a more convenient basis before slicing.

The number of quantized states in the codebook is deliberately while learning the vector-quantized representations to encourage representations to be parsimonious and avoid “hiding” nuisance covariates, which may include speaker-identifiable information, in small details.

In this example the product quantizer comprises of 3 vector quantizers each of dimension 10 with an independent codebook of size 32, giving a maximum number of states of 32×32×32=around 32.8k per audio-word. A decay of γ=0.99 was chosen for all quantizers and the commitment loss is weighted by α=0.5. The linear layers have dimensionality 30.

Transformer Encoder

The product-quantized vector sequence is fed to a standard Transformer encoder architecture (Vaswani, A., Attention is all you need. arXiv preprint arXiv:1706.03762, 2017). Fixed sine/cosine positional embeddings are used to allow the encoder to exploit position information. By contextualizing prosody representations it is possible to make representations with weaker cross-temporal interactions. Context-aware representations of time-series often make better predictions and therefore contextualization may be used to make stronger prosodic representations for predictions. Contextualisation also allows for disentangling representations from time, which facilitates audio-linguistic representation learning.

In this specific example The Transformer encoder has 12 layers, 12 attention heads, inner (FFN) dimension 3,072, embedding size 768, ReLU activation and a 10% dropout probability. The positional encoding is implemented as per the BERT (Devlin et al., 2018) paper. Since prosody temporal interactions are relatively short compared to language, the sequence length is restricted to 32 words. During pretraining, we also require a minimum sequence length of 16 words. We train using K=9 distractors.

Probing and De-Identification

For explainability purposes, it is desirable to measure how well a feature is represented in a given representation. One approach used within the present invention is to use the prequential (or online) approach to minimum description length (MDL) to quantify the regularity between representations and labels (see for example Voita, E. and Titov, I. Information-theoretic probing with minimum description length. arXiv preprint arXiv:2003.12298, 2020).

MDL measures the number of bits required to transmit the labels given the representations. If a feature is highly extractable from a given representation, a model trained to detect said feature will converge quickly, resulting in a small MDL. Computing the MDL using the prequential approach requires sequential training and evaluation. The train set is partitioned into timesteps and the probe is trained one one set and evaluated on another. The codelength is calculated as per (Voita & Titov, 2020 cited above).

The method is further adapted to derive an information theoretic definition of speech identifiability. Following the literature (Tomashenko, N., et al. Introducing the voiceprivacy initiative. arXiv preprint arXiv:2005.01387, 2020), this is considered as a number of binary speaker verification trials but, instead of using equal error rate or log-likelihood-based metrics, the de-identification ratio of a set of trial representations is defined with respect to enrolment representations as the inverse of the compression ratio of the theoretical minimum description length to transmit the data using a prequential approach:

The rationale is that a shorter MDL means that the verification task is easier given the two representations. This improves upon prior work, which assumes a fixed model (usually a probabilistic LDA) by taking into account the effort required to perform verification as well as the performance on the task. Real attackers could have access to sophisticated models and arbitrary computational resources to compare speech representations, motivating this approach.

Training of Model

Recent work on Transformer architectures has demonstrated the importance of using large datasets for pretraining, and many models improving over the state of the art have used increasingly large datasets. The methods of the present invention have been tested by pretraining the models on a new dataset, the Colossal Audio-linguistic Corpus (CALC), a large word-aligned audio-linguistic dataset of natural speech with matching audio and text modalities. CALC is composed of five datasets wrangled into a common format, chosen based on their size, prior use in the literature, and whether they contain natural speech.

The model is trained using a self-supervised contrastive signal, followed by assessing performance on a supervised task. The representations are not fine-tuned on the supervised task to preclude the model from pulling out new, perhaps identifiable, information from the raw audio during supervision. The model is pre-trained using a BERT-like masking paradigm, with a contrastive self-supervised signal similar to wav2vec 2.0. The pretraining task is to identify the correct latent prosody representation in the presence of a number of distractors sampled from other masked timesteps. We mask timesteps with a fixed probability and consider a two-part loss function: a contrastive loss and a commitment loss. The contrastive loss for selecting the true latent prosody representation amongst a set of distractors, which are uniformly sampled from other masked timesteps of the same sample. The commitment loss penalizes discrepancies between the quantizer inputs and outputs to encourage robustness. The commitment loss is averaged over the N constituent vector quantizers in our product quantizer. In lieu of a codebook loss, exponential moving average updates are used for the codebook as per (Oord et al., 2017). For training on downstream tasks, a simple two layer feed-forward network (FFN) with hidden size 256, batch size 256, ReLU activations, dropout with probability 30% using the Adam optimizer (Kingma & Ba, 2014) with learning rate α=10⁻³ and default parameters β₁=0.9, β₂=0.99. A final sigmoid activation and binary cross-entropy loss is used. The input dimension varies across the different representations. We train these models for 20k steps and use the last model states to report performance on the downstream tasks.

During training 30% of all prosody tokens are uniformly masked. The learning rate is warmed up linearly from 0 to a maximum of 1.5×10⁵ at 10k steps before linearly decaying it. The model trains for 250k steps using the AdamW optimizer (Loshchilov & Hutter, 2017). A batch size of 128 samples and the model is trained on a single V100 GPU for 2.3 days.

The results of testing showed that the representations obtained using the method and model according to the present invention outperformed prior art representations. In particular, the representations obtained using the above described method were compared against those of four recent audio representation learning models: Mockingjay (Liu et al., 2020), vq-wav2vec (Baevski et al., 2019), wav2vec 2.0 (Baevski et al., 2020) and TRILL (Shor et al., 2020). In particular a simulation was run to test each models ability to uniquely identify a correct speaker by using a speech representation from each of a group of N people and seeking to find out from whom a separate target speech representation came. For simplicity, it is assumed that the model outputs a binary value, the trials are independent and that the model must uniquely identify the correct person on the basis of the speech representation provided. For N=10 people, the representations obtained using the method of the present invention had a probability of correctly identifying the speaker of 1.58%, compared to: TRILL 5.10%, vq-wav2vec 24.8%, wav2vec-2.0 37.7% and Mockingjay 44.3%.

1. A computer-implemented method of obtaining de-identified representations of audio speech data for use in a speech analysis task, where the audio speech data comprises a raw audio signal, the method comprising:
pre-processing the audio speech data to remove timbral information by downsampling the audio speech data, such that the pre-processed audio speech data comprises a downsampled raw audio signal; and
encoding sections of the pre-processed audio speech data into audio representations by inputting sections of the pre-processed audio data into a prosody encoder, the prosody encoder comprising a machine learning model trained using self-supervised learning to map sections of the pre-processed audio data to corresponding audio representations.

2. The computer-implemented method of claim 1 wherein pre-processing the audio speech data comprises:
downsampling the audio speech data at a rate of less than 1000 Hz, preferably between 400 Hz and 600 Hz.

3. The computer-implemented method of claim 1 wherein training the machine learning model using self-supervised learning comprises withholding part of the input data and training the machine learning model to predict the withheld part of the input data.

4. The computer-implemented method of claim 1 wherein the prosody encoder comprises a machine learning model trained using a masked language modelling objective.

5. The computer-implemented method of claim 1 wherein the prosody encoder comprises a machine learning model trained to map sections of the pre-processed audio data to corresponding audio representations with no access to the linguistic information.

6. The computer-implemented method of claim 1 comprising:
splitting the audio speech data into audio words, the audio words comprising variable-length sections of the audio speech data, each containing one spoken word of the audio speech data,
wherein the model is trained to map input audio words to corresponding quantised representations encoding prosodic information of the audio word.

7. The computer-implemented method of claim 6 wherein the audio words include a period of silence preceding the spoken word, preferably wherein the period is up to 2 seconds in length.

8. The computer-implemented method of claim 1 comprising normalising the average pitch of voiced sections of the audio speech data to a predetermined frequency.

9. The computer-implemented method of claim 1 wherein encoding sections of the pre-processed audio speech data into audio representations comprises:
encoding sections of the pre-processed audio speech data into quantised audio representations, wherein the prosody encoder comprises a machine learning model trained to map sections of the pre-processed audio data to corresponding quantised audio representations.

10. The computer-implemented method of claim 9 wherein encoding sections of the pre-processed audio speech data into quantised audio representations comprises:
encoding each section of pre-processed audio speech data into one of a fixed number of quantised audio representations, where the fixed number of quantised audio representations is between 100 and 100,000.

11. The computer-implemented method of claim 1 wherein the prosody encoder comprises:
a first machine learning model trained to encode sections of the pre-processed audio data into corresponding non-quantised audio representations; and
a second machine learning model trained to quantise each audio representations output from the first machine learning model into one of a fixed number of quantised audio representations.

12. The computer-implemented method of claim 11 wherein the first machine learning model is trained to encode sections of the pre-processed audio data into corresponding non-quantised audio-representations and the second machine learning model is trained to perform vector quantisation on the non-quantised audio representations output by the first machine learning model.

13. The computer-implemented method of claim 11 wherein the first machine learning model comprises a temporal convolutional neural network.

14. The computer-implemented method of claim 11 wherein the second machine learning model is trained to perform product quantisation on each non-quantised audio representations.

15. The computer-implemented method of claim 11 further comprising:
inputting a sequence of quantised audio representations into a contextualisation model, the contextualisation model comprising a machine learning model trained to encode the quantised audio representations into corresponding contextualised audio representations which encode information relating to their context within the sequence.

16. The computer-implemented method of claim 15 wherein the contextualisation model comprises a Transformer model.

17. The computer-implemented method of claim 15 wherein the contextualisation model is configured to consider interactions between two quantised word representations in the sequence only up to a maximum number of separating words between the two quantised word representations, where the maximum number of separating words is within the range 10 to 1000 words, preferably 20 to 120 words.

18. The computer-implemented method of claim 15 wherein the prosody encoder and the contextualisation model are trained using self-supervised learning using a masked language modelling objective.

19. A computer-implemented method of performing speech analysis to determine or monitor a health condition of a speaker, the method using audio speech data comprising a raw audio signal, the method comprising:
obtaining de-identified audio representations of the audio speech data using the method of claim 1; and
inputting the audio representations in a task-specific machine learning model trained to map the de-identified audio representations to an output associated with a health condition.
